(19) United States
(12) Patent Application Publication
(10) Pub. No.: US 2025/0061985 A1
(43) Pub. Date: Feb. 20, 2025

(54) DIAGNOSTIC LABORATORY SYSTEMS AND METHODS OF UPDATING

(71) Applicant: Siemens Healthcare Diagnostics Inc., Tarrytown, NY (US)

(72) Inventors: Venkatesh NarasimhaMurthy, Hillsborough, NJ (US); Vivek Singh, Princeton, NJ (US); Yao-Jen Chang, Princeton, NJ (US); Benjamin S. Pollack, Jersey City, NJ (US); Ankur Kapoor, Plainsboro, NJ (US); Rayal Prasad, Princeton, NJ (US)

(73) Assignee: Siemens Healthcare Diagnostics Inc., Tarrytown, NY (US)

(21) Appl. No.: 18/723,535

(22) PCT Filed: Mar. 2, 2023
(86) PCT No.: PCT/US2023/063613

§ 371 Date: Jun. 24, 2024

Related U.S. Application Data
(60) Provisional application No. 63/268,788, filed on Mar. 2, 2022.

(51) Int. Cl. G16H 10/40

(52) U.S. Cl. CPC G16H 10/40

(57) ABSTRACT

Diagnostic laboratory systems, methods of operating diagnostic laboratory systems, and methods of updating diagnostic laboratory systems are disclosed. A method of operating a diagnostic laboratory system includes identifying data in the system as identified data: identifying change in a data distribution of the identified data; aggregating data instances pertaining to the change in the data distribution; selecting a subset of the aggregated data to update a processing algorithm in the system; and updating the processing algorithm using the subset of the aggregated data. Other systems and methods are disclosed.

### CROSS REFERENCE TO RELATED APPLICATION

This application claims the benefit of U.S. Provisional Patent Application No. 63/268,788, entitled “DIAGNOSTIC LABORATORY SYSTEMS AND METHODS OF UPDATING” filed Mar. 2, 2022, the disclosure of which are hereby incorporated by reference in their entirety for all purposes.

### FIELD

Embodiments of the present disclosure relate to diagnostic laboratory systems and methods of updating processing algorithms in the diagnostic laboratory systems.

### BACKGROUND

Diagnostic laboratory systems include instruments that conduct clinical chemistry or assays to identify analytes or other constituents in biological specimens such as blood serum, blood plasma, urine, interstitial liquid, cerebrospinal liquids, and the like. The systems use algorithms to analyze data input to the systems and generated by the instruments. As components in the systems change and input parameters to the systems change, the algorithms need to be updated. Updating and validating the algorithms are time-consuming and costly. Therefore, a need exists for systems and methods that efficiently update the algorithms.

### SUMMARY

According to a first aspect, a method of operating a diagnostic laboratory system is provided. The method includes identifying data in the system; identifying change in a data distribution of the identified data; aggregating data instances pertaining to the change in the data distribution; selecting a subset of the aggregated data to update a processing algorithm in the system; and updating the processing algorithm using the subset of the aggregated data.

According to a second aspect, a diagnostic laboratory system is provided. The system includes a computer comprising: a processing algorithm configured to analyze data; and a program configured to: identify data in the system as identified data; identify change in a data distribution of the identified data; aggregate data instances pertaining to the change in the data distribution; select a subset of the aggregated data to update the processing algorithm in the system; and update the processing algorithm using the subset of the aggregated data.

In another aspect, a method of operating a diagnostic laboratory system is provided. The method includes identifying data in the system as identified data; identifying change in a data distribution of the identified data; aggregating data instances pertaining to the change in the data distribution; selecting at least two subsets of data; evaluating at least one algorithm parameter of a processing algorithm using a first subset of data; evaluating at least one of the algorithm parameters using a second subset of data; selecting one or more algorithm parameters in response to the evaluating; and updating the processing algorithm using the one or more selected algorithm parameters.

Still other aspects, features, and advantages of this disclosure may be readily apparent from the following description and illustration of a number of example embodiments, including the best mode contemplated for carrying out the disclosure. This disclosure may also be capable of other and different embodiments, and its several details may be modified in various respects, all without departing from the scope of the disclosure. This disclosure is intended to cover all modifications, equivalents, and alternatives falling within the scope of the claims and their equivalents.

### BRIEF DESCRIPTION OF THE DRAWINGS

The drawings, described below, are provided for illustrative purposes, and are not necessarily drawn to scale. Accordingly, the drawings and descriptions are to be regarded as illustrative in nature, and not as restrictive. The drawings are not intended to limit the scope of the disclosure in any way.

FIG. 1 illustrates a block diagram of a diagnostic laboratory system including a plurality of instruments according to one or more embodiments.

FIG. 2A illustrates a side elevation view of a capped specimen container containing a specimen, wherein the specimen container is configured to be transported throughout a diagnostic laboratory system according to one or more embodiments.

FIG. 2B illustrates a side elevation view of an uncapped specimen container containing a specimen, wherein the specimen container is configured to be moved to at least one instrument in a diagnostic laboratory system according to one or more embodiments.

FIG. 3 illustrates a top plan view an imaging instrument of a diagnostic laboratory system including three imaging devices according to one or more embodiments.

FIG. 4 illustrates a block diagram of an aspiration and dispensing instrument of a diagnostic laboratory system according to one or more embodiments.

FIG. 5 is a graph illustrating pressure traces of a pipette assembly of an aspiration and dispensing instrument showing a pressure trace of an original system, a pressure trace after a component has been replaced, and a pressure trace of a malfunctioning aspiration module according to one or more embodiments.

FIG. 6 is a flowchart illustrating a method of operating a diagnostic laboratory system according to one or more embodiments.

FIG. 7 is a flowchart illustrating a method of operating a diagnostic laboratory system according to one or more embodiments.

### DETAILED DESCRIPTION

Diagnostic laboratory systems (systems) conduct clinical chemistry and/or assays to identify analytes or other constituents in biological specimens such as blood serum, blood plasma, urine, interstitial liquid, cerebrospinal liquids, and the like. Instruments, subsystems, and other components of the systems must operate precisely in order to conduct accurate assays. In addition, analytical chemicals used in the systems have to be within specific limits to perform accurate assays. In some circumstances, when new components and/or new chemicals are introduced to the systems, processing algorithms that operate the systems may have to be updated.

The systems may include individual instruments that conduct the clinical chemistry and/or assays to identify analytes or other constituents in the specimens. The specimens are typically stored in specimen containers and are transported to specific instruments within the systems for processing and/or testing.

Some systems perform pre-analytical specimen and/or specimen container processing. For example, some instruments may perform centrifugation of specimens to separate specimen constituents. Some instruments may perform removal of caps from tube portions of the specimen containers to enable access to specimens located in the tube portions. Some instruments may perform aliquot preparation. Other instruments may pre-screen specimens for HIL (hemolysis (H), icterus (I), and/or lipemia (L)) and/or the presence of artifacts in the specimens such as a clot, bubble, or foam. Some instruments may use one or more sensors, such as imaging devices, coupled to a computer to perform the above-described analyses. For example, imaging devices may capture images of the specimens and/or the specimen containers and the computer may analyze the image data generated by the imaging devices to perform the above-described analyses.

In some embodiments, the systems include instruments containing clinical chemistry and/or assay modules configured to perform analytical tests on the specimens. The testing may involve reactions that generate changes, such as fluorescence or luminescence emissions that may be read to determine a presence and/or a concentration of an analyte or other constituent contained in the specimens. Some instruments may include one or more sensors, such as one or more imaging devices coupled to a computer, wherein the computer analyzes image data generated by the one or more imaging devices to determine the concentration and/or presence of analytes.

The systems may include a plurality of sensors, which may include the aforementioned imaging devices. Some systems may include one or more pressure sensors configured to measure aspiration and/or dispense pressure, such as pressure in a pipette assembly during aspiration and/or dispense processes. Temperature sensors may measure temperatures of specimens, analytes, incubation devices, machinery, and other components. Voltage sensors may measure voltages of various machinery and/or specimens. Acoustic sensors and vibration sensors may measure acoustic noise and vibration, respectively, of machinery and other components within the systems.

Collision sensors may generate data indicating the occurrence of collisions within the systems. For example, collision sensors may generate data indicating collision of robotic arms and other moving components within the systems. Distance sensors and proximity sensors may determine relative locations of moving components, including specimen containers, within the systems. Tactile sensors, which may be implemented as capacitive sensors, may generate data (e.g., signals) when components within the systems contact.

Processing algorithms that operate the systems and the individual instruments may be engineered and evaluated on large datasets (e.g., validation datasets) to be operable under a variety of conditions. The systems are often deployed for several years during which time changes in data distribution and input parameters, such as tube types, inevitably occur. These changes may be due to changes in hardware, software, specimen containers, and other items. The changes also may be made to accommodate manufacturer changes, changes in labeling/barcode designs affixed to specimen containers, and changes to assay protocols. The changes may be due to other reasons.

When these changes are made, the processing algorithms that operate the systems or individual instruments may be updated to operate properly with the changes. Disclosed herein are systems and software that can update and/or adapt to accommodate changes. For example, the systems and/or software described herein may include a program, such as artificial intelligence (AI or AI models) that can update the processing algorithms. Artificial intelligence includes, but is not limited to neural networks, including convolutional neural networks (CNNs), deep learning, regenerative networks, and other machine learning and artificial intelligence algorithms. AI may not be lookup tables. Rather, AI may include AI models that are trained to recognize (e.g., characterize) a variety of different data. A lookup table, on the other hand, is only able to identify data that is specifically in the lookup table.

The program may determine that a change in a data distribution in data input and/or generated by a system has occurred, which may trigger the system to update or retrain itself. In some embodiments, a user may cause the system to update or retrain itself. In some embodiments, the processing algorithms may be updated to reflect changes that are specific to the sites where the systems are located. For example, a user may change hardware or input parameters unique to testing on a unique system.

A program (e.g., software) or a plurality of programs, such as AI algorithms, may identify changes in the data distribution or usage of the system or any sub-system (e.g., instrument). The program may aggregate data instances, both input and output, pertaining to the changes in data distribution. The program may select a subset of data, such as image data, text data, or sensor data along with its ground truth, to update the system (e.g., the processing algorithms). In some embodiments, the program may enable a user to select the subset of data. The program may update system parameters (e.g., processing algorithm parameters) using the selected data instances. In some embodiments, the program may then generate a report based on the updates, wherein the report may be compliant with rules of one or more regulatory agencies having jurisdiction over the systems.

In some embodiments, the processing algorithm may be continuously updated. For example, the program may continuously analyze input and/or output data to determine if updates and/or retraining of the processing algorithm is required. In some embodiments, the program may periodically analyze the data to determine if updates are necessary. In some embodiments, the program may update the processing algorithm periodically.

In some embodiments, the aggregated data may include any information provided as input to the system including assay types, date of the assay, and/or time that one or more assays are performed. In some embodiments, the data may include one or more measurements generated by an instrument, such as, optical, acoustic, photometric, and/or temperature data. The data may be one-dimensional, two-dimensional, or three-dimensional. In some embodiments, the data may be images captured by one or more sensors, univariate or multivariate time series data, text labels, and/or system logs.

In some embodiments, the change in data distribution may include or be related to changes in input data statistics compared to population statistics of the data on which the system was trained. In some embodiments changes in data distribution may be changes in usage of the system and may relate to the changes in one or more user interactions with the system. User workflows may be traced over time to determine changes in usage trends.

In some embodiments, the data aggregation may include aggregated input data to a subsystem (e.g., an instrument) as well as expected output data. The expected output data of a subsystem may be obtained using measurements generated by another subsystem or may be obtained by user interaction with the system. In some embodiments, the data may be aggregated locally on a system storage device, an external device coupled to the system, or cloud storage. In some embodiments, a selection of the dataset is obtained by automatically selecting a smaller set of samples that spans variations observed in the data but does not have similar samples repeated.

In some embodiments, similarity of the samples may be determined by comparing input measurements to the subsystem or by using at least one statistical method. In some embodiments, similarity of the samples may be determined by AI algorithms, such as by machine learning. In some embodiments, the selection of data includes selecting at least two subsets of data, wherein, the first subset may be used to compute system or algorithm parameters and the second subset may be used to evaluate and compare various parameter sets and determine the best data set for use in the processing algorithm. In some embodiments, the processing algorithm may be a machine learning algorithm such as a convolutional neural network, for example. In some embodiments, the updated system parameters may be determined using a conjunction of the aggregated data as well as a prior training dataset stored on the system and/or stored remotely. Data related to specific patient and/or site information may be removed from the data so that the data is anonymous.

Notifications of the updates may be provided (e.g., displayed) on a user interface associated with the system or with an instrument that is updated. The update may be logged into software or other records associated with the updated system. The notice may include a summary of the changes and/or improvements to the system. In some embodiments, the user may be notified to enable or disable the update.

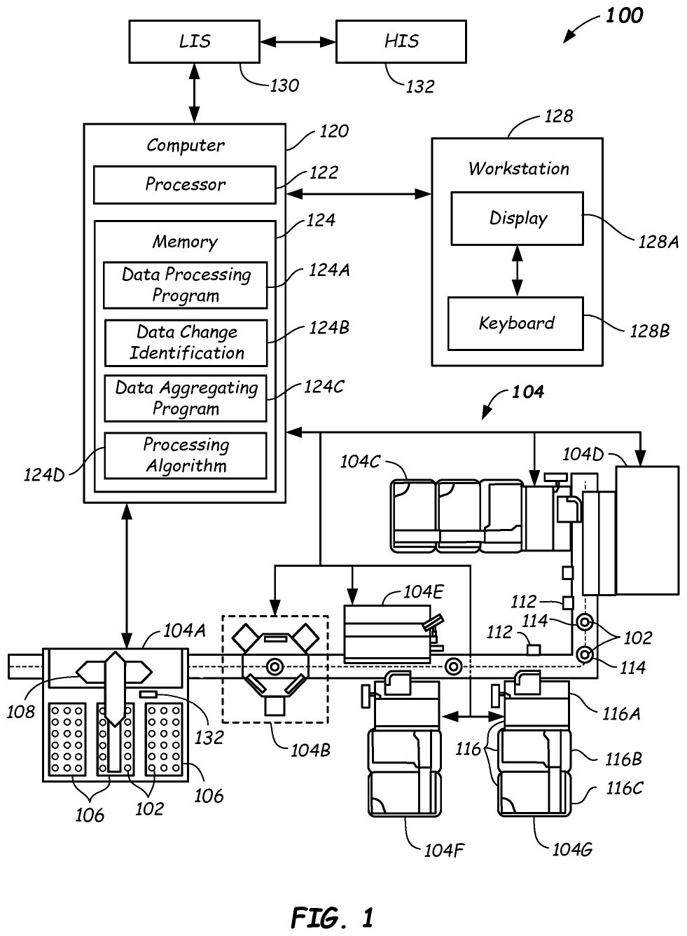

Further details of systems that update processing algorithms, methods of operating systems, and methods of updating processing algorithms are further described with reference to FIGS. 1-7 herein.

Reference is now made to FIG. 1, which illustrates an example embodiment of an automated diagnostic laboratory system 100 configured to process and/or analyze biological specimens stored in specimen containers 102. The specimen containers 102 may be any suitable containers, including transparent or translucent containers, such as blood collection tubes, test tubes, sample cups, cuvettes, or other containers capable of containing specimens and/or allowing imaging of the specimens contained therein. The specimen containers 102 may have different sizes and may have different cap colors and/or cap types.

The system 100 may include a plurality of instruments 104 that are configured to process the specimen containers 102 and/or specimens located therein. The specimen containers 102 may be received at the system 100 at a load/unload instrument 104A that may include one or more racks 106 and a robot 108, wherein the specimen containers 102 may be loaded into the racks 106. The robot 108 may transport the specimen containers 102 between the racks 106 and carriers 114 located on a track 112. The specimen containers 102 may be transported throughout the system 100, such as to and from the instruments 104 on the track 112 by the carriers 114. As described herein, one or more instruments 104 or components located therein may identify the types of specimen containers 102 present in the system 100 and may configure the robot 108 to handle the specimen containers 102 based on their types.

Processing of the specimens and/or the specimen containers 102 may include preprocessing or pre-screening of the specimens and/or the specimen containers 102 prior to analysis by one or more of the instruments 104. Other ones of the instruments 104 may perform the analyses of the specimens described herein. The embodiment of the system 100 depicted in FIG. 1 includes seven instruments 104, which are referred to as a first instrument or the load/unload instrument 104A and second through seventh instruments 104B-104G, respectively. Other embodiments of systems may include different numbers of instruments.

One or more of the instruments 104 may include one or modules 116, such as preprocessing and/or analyzer modules. Reference is made to the seventh instrument 104G that includes three modules 116. The seventh instrument 104G is representative of some of the other instruments 104. Other ones of the instruments 104 may include more or fewer individual modules. In the embodiment of FIG. 1, a first module 116A may be a preprocessing module, for example, that processes the specimen containers 102 and/or specimens located therein prior to analyses by analyzer modules. A second module 116B and a third module 116C may be analyzer modules that analyze specimens as described herein.

The system 100 may include or be coupled to a computer 120 that may be configured to operate and/or communicate with the instruments 104. In some embodiments, the computer 120 may be configured to analyze data associated with one or more of the instruments 104. The data may include data input to the instruments 104, data output from the instruments 104, and/or data generated by the instruments 104. In the embodiment of FIG. 1, the computer 120 may be connected to individual ones of the instruments 104. In some embodiments, the computer 120 may be coupled to one or more workstations (not shown) coupled to one or more of the instruments 104. In some embodiments, the computer 120 may be configured to transmit computer-readable instructions and/or computer code to individual ones of the instruments 104. In some embodiments, the computer 120 may be implemented in one or more of the instruments 104.

The computer 120 may include one or more programs and/or components implemented as hardware and/or software. Programs other than those described herein may be present in the computer 120. The computer 120 may include a processor 122 that may be configured to execute computer code (e.g., the programs), such as computer code stored in memory 124. In some embodiments, the processor 122 may be configured to execute computer code from other sources, such as external sources and/or the cloud.

The memory 124 may store or access one or more programs, which are described herein as being stored in the memory 124. Some of the programs may be configured to operate one or more of the instruments 104. Although the programs are described as individual programs, in some embodiments the programs may be implemented as a single program. One or more of the programs may operate using artificial intelligence (AI) algorithms. The memory 124 may include a data processing program 124A that may be configured to identify data in at least one of the instruments 104. For example, the data processing program 124A may identify data input to the system 100 and/or one or more of the instruments 104. The data processing program 124A may also identify data generated by and/or located in one or more of the instruments 104. The data processing program 124A may also analyze data output by one or more of the instruments 104.

In some embodiments, the memory 124 may include a data change identification program 124B that may be configured to identify changes in data distribution in the data identified by the data processing program 124A. The changes in data (data distribution) may be data input to the system 100 and/or output by the system 100. The changes in data distribution may be changes in data input, output, located in, and/or generated by one or more of the instruments 104. In some embodiments, the memory 124 may include a data aggregating program 124C. In some embodiments, the data aggregating program 124C may aggregate data, such as the data identified by the data change identification program 124B.

In some embodiments, the memory 124 may include a processing algorithm 124D. In some embodiments, the memory 124 may include more than one processing algorithm that may be implemented as a single processing algorithm, such as the processing algorithm 124D. The processing algorithm 124D may include computer code configured to analyze data, such as data generated by the instruments 104. In some embodiments, the processing algorithm 124D may process data using AI algorithms that analyze the data described herein. As described herein, the processing algorithm 124D may be updated as described herein. For example, in some embodiments, one or more AI algorithms may be updated.

The computer 120 may be coupled to or implemented in a workstation 128. The workstation 128 may enable user interaction with the system 100 and/or one or more of the instruments 104. The workstation 128 may include a display 128A and/or a keyboard 128B that enable a user to interface with the system 100 and/or individual ones of the instruments 104.

The computer 120 may, by way of one or more of the components in the system 100, control movement of the carriers 114 between different ones of the instruments 104 by way of the track 112. One or more of the instruments 104 may be in communication with the computer 120 through a network, such as a local area network (LAN), wide area network (WAN), or other suitable communication network, including wired and wireless networks.

In some embodiments, the system 100 may be coupled to a laboratory information system (LIS) 130 that may determine how specimens are to be tested by the system 100. In some embodiments, the LIS 130 may be implemented in the computer 120. The LIS 130 may be coupled to a hospital information system (HIS) 132 that may receive specific assay orders for specific specimens. The HIS 132 may also receive assay results after assays are performed on the specific specimens.

The systems and methods disclosed herein are configured to detect changes in at least data or system usage. The systems and methods enable the processing algorithm 124D, other programs, and/or programs in individual ones of the instruments 104 to update/adapt to accommodate the changes. Various data monitoring and updating methods are described herein.

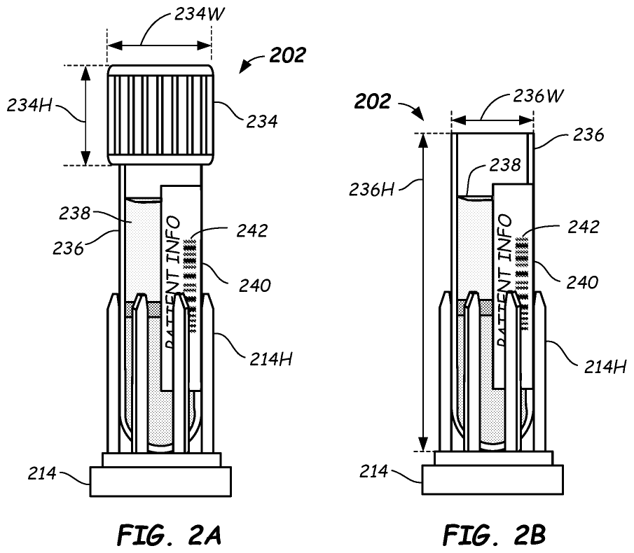

Additional reference is made to FIGS. 2A-2B, which illustrate a specimen container 202 being carried in a carrier 214. The embodiment of the specimen container 202 of FIG. 2A includes a cap 234 and the embodiment of the specimen container 202 of FIG. 2B does not include a cap. The cap 234 of FIG. 2A is only one of many types of caps that may seal the specimen container 202. As described herein, one or more of the instruments 104 (FIG. 1) may determine the cap type.

The specimen container 202 and/or the carrier 214 may be similar or identical to at least one of the specimen containers 102 and/or carriers 114 of FIG. 1. The specimen container 202 is one of many different types of specimen containers that may be used in the system 100. The types of specimen containers used in the system 100 may be varied and may change over time. As described herein one or more of the instruments 104 may be configured to identify the types of specimen containers 102 present in the system 100. For example, components in one or more of the instruments 104 may include one or more sensors, such as an imaging device, that generates image data representative of the specimen container 202. The data processing algorithm 124D or other programs in the memory 124 may use algorithms such as AI algorithms to identify (e.g., classify) the specimen container 202 (e.g., the type of specimen container).

As described herein, the system 100 may determine whether the specimen container 220 has the cap 234 attached to a tube 236. In some embodiments, the system 100 may also determine the type of specimen container and/or the tube 236. For example, the system 100 may determine if the specimen container 202 is the specimen container type or tube type shown in FIGS. 2A-2B. Other types of specimen containers, such as tube top sample cups (TTSCs), may be detected by the system 100. By identifying the type of the specimen container 202 and/or the type of tube 236, the system 100 can instruct transportation devices, such as the robot 108, to optimally handle the specimen containers 102.

In the embodiment of FIG. 2A, the specimen container 202 has the cap 234 attached to the tube 236. During processing by one or more of the instruments 104, the cap 234 is removed to access a specimen 238 located in the tube 236 as shown in FIG. 2B. The shape and/or color of the cap 234 may indicate the type of tube 236. The shape and/or color of the cap 234 also may indicate the type of specimen 238 and any chemicals other than the specimen 238 that are located in the tube 236. Caps on different specimen containers may be of different types and/or colors (e.g., red, royal blue, light blue, green, grey, tan, yellow, or color combinations), which may have meaning in terms of specific tests the specimen container 202 is used for. For example, the cap configuration may indicate a type of additive included in the tube 236. For example, chemicals used to process the specimen 238 may be added to the tube 236 prior to the specimen 238 being added to the tube 236. The cap type may also indicate whether the tube 236 includes a gel separator. In some embodiments, the system may determine the cap type using a characterization method described herein, such as by the processing algorithm 124D. In the embodiment of FIG. 2A, the cap 234 has a cap height 234H and a cap width 234W. The tube 236 shown in FIG. 2B has a tube width 236W and a tube height 236H.

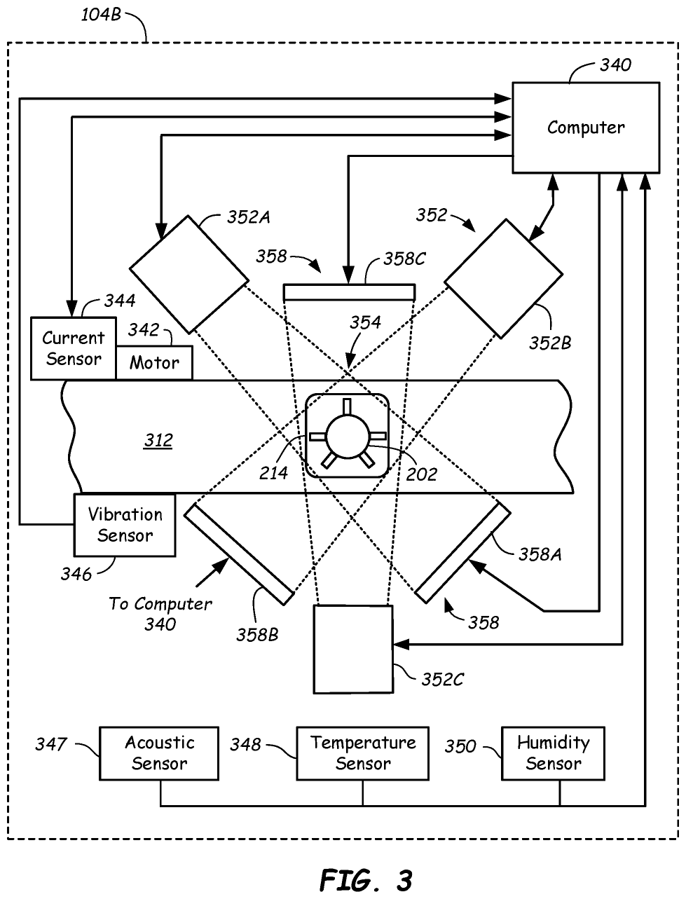

The second instrument 104B may include an imaging device (e.g., imaging devices 352-FIG. 3) that captures one or more images of the specimen container 202 with or without the cap 234. Image data generated by the imaging device may be processed by the data processing program 124A and/or the processing algorithm 124D as described herein. The data processing program 124A, the processing algorithm 124D, or other programs may determine certain features, such as the cap height 234H, cap width 234W, color of the cap 234, tube height 236H, and/or tube width 236W.

Data pertaining to the specimen container 202 may be data input to the system 100 and/or one or more of the instruments 104. In some embodiments, the above-described image data generated by the imaging device may be data input to the system 100 and/or one or more of the instruments 104. In some embodiments, the above-described image data may be data present within one or more of the instruments 104. In some embodiments, one or more of the instruments 104, other than the second instrument 104B, may be an imaging device or include an imaging device that outputs image data. Thus, the above-described image data may be data output from one or more of the instruments 104. As described in greater detail below, the data change identification program 124B may determine whether the image data generated by the one or more imaging devices has changed over time. The change in image data may constitute a change in the data distribution.

As shown in FIGS. 2A-2B, the specimen container 202 is located in a carrier 214. In some embodiments, the carrier 214 may be unique to specific ones of the specimen containers 102, such as the specimen container 202. The carrier 214 may be representative of the carriers 114 (FIG. 1). The carrier 214 may include a holder 214H configured to hold the specimen container 202 in a defined upright position and orientation. In some embodiments, the holder 214H may include a plurality of fingers or leaf springs that secure the specimen container 202 in the carrier 214. The carriers 114 may be changed over time to accommodate different types of the specimen containers 102. These changes may be identified by the data change identification program 124B. In some embodiments, the images of the carrier 214 may be captured and processed as described with reference to the specimen container 202. Accordingly, the carrier 214 and/or images of the carrier 214, including text data, may be the data input to, output from, and/or within one or more of the instruments 104.

The specimen container 202 may be provided with at least one label 240 that may include identification information 242 (i.e., indicia) thereon, such as a barcode, alphabetic characters, numeric characters, or combinations thereof. The identification information 242 may include or be associated with data provided to the system 100 by the LIS 130. The data may be in the form of a database stored in the memory 124 and may include information referred to as text data such as patient information, including name, date of birth, address, and/or other personal information as described herein. The data may also include other text data, such as tests to be performed on the specimen 238, time and date the specimen 238 was obtained, medical facility information, and/or tracking and routing information. Other text data may also be included.

Additional reference is made to FIG. 3, which is a top plan view of the second instrument 104B with a top cover removed. The second instrument 104B may be configured to capture images of the specimen container 202, the specimen 238, and/or the carrier 214. Capturing images generates image data representative of the items captured. The second instrument 104B may be referred to as a quality check module that checks the specimen 238 prior to analyses by other ones of the instruments 104.

The second instrument 104B may include a computer 340 that may control and/or communicate with components within the second instrument 104B. The computer 340 may communicate with and receive instructions from the computer 120 (FIG. 1). The communications enable transmission of data generated by the second instrument 104B to the computer 120. The communications also may instruct the computer 340 to cause imaging devices within the second instrument 104B to capture images of the specimen 238, the specimen container 202, and/or the carrier 214.

In the embodiment of FIG. 3, the specimen container 202, via the carrier 214, is transferred into the second instrument 104B via a track 312. The track 312 may be a portion of the track 112 (FIG. 1). In other embodiments, the track 312 may be a unique track located within the second instrument 104B wherein the specimen container 202 has been transferred to the track 312 via a transport device (not shown) such as a robot or the like. In some embodiments, the track 312 may be moveable by a motor 342 that is controlled by instructions generated by the computer 340. A current sensor 344 may measure current drawn by the motor 342 and transmit the measured current to the computer 340. In some embodiments, the second instrument 104B may include a vibration sensor 346 that may sense vibration in the second instrument 104B. In some embodiments, the vibration sensor 346 may measure vibration associated with the track 312 and/or movement of the specimen container 202 within the second instrument 104B. The vibration sensor 346 may generate vibration data that is transferred to the computer 340.

The second instrument 104B may include other sensors. In the embodiment of FIG. 3, the second instrument 104B includes an acoustic sensor 347, a temperature sensor 348, and a humidity sensor 350. The acoustic sensor 347 is configured to measure noise and generate noise data that may be transmitted to the computer 340. The temperature sensor 348 is configured to measure temperature within the second instrument 104B and generate temperature data that may be transmitted to the computer 340. The humidity sensor 350 is configured to measure humidity within the second instrument 104B and generate humidity data that may be transmitted to the computer 340.

The second instrument 104B may include one or more imaging devices 352. The imaging devices 352 are referred to individually as a first imaging device 352A, a second imaging device 352B, and a third imaging device 352C. The imaging devices 352 may be configured to capture images of the specimen container 202, the specimen 238, and/or the carrier 214 at an imaging location 354 from multiple viewpoints. While three imaging devices 352 are shown in FIG. 3, optionally, one, two, four, or more imaging devices can be used.

Each of imaging devices 352 may be individually controlled by the computer 340. For example, the computer 340 may transmit instructions to individual ones of the imaging devices 352 that cause the individual imaging device to capture an image. Each of the captured images may be processed by the processing algorithm 124D (FIG. 1) or other programs according to one or more embodiments described herein. The imaging devices 352 may be any suitable devices configured to capture digital images. In some embodiments, each of the imaging devices 352 may be a conventional digital camera capable of capturing pixelated images.

The second instrument 104B may include one or more light sources 358 that are configured to illuminate the imaging location 354. Thus, the light sources 358 may be configured to illuminate the specimen container 202, the specimen 238, and/or the carrier 214 during image capturing. In the embodiment of FIG. 3, the second instrument 104B includes three light sources 358, which are referred to individually as a first light source 358A, a second light source 358B, and a third light source 358C. In some embodiments, the light sources 358 may provide front lighting of the imaging location 354 and may be individually controlled by instructions generated by and/or transmitted from the computer 340.

In some embodiments, the characterizations associated with data generated by sensors in the second instrument 104B may include determining a presence of and/or an extent or degree of hemolysis (H), icterus (I), and/or lipemia (L) contained in the specimen 238 (FIG. 2B). In some embodiments, the characterization may determine whether the specimen 238 is normal (N). If the specimen 238 is found to be normal (N), the specimen 238 may continue on the track 312 where the specimen 238 may be analyzed (e.g., tested) by the one or more of the instruments 104 (FIG. 1). Other pre-processing operations may be conducted on the specimen 238 and/or the specimen container 202. The characterization and/or pre-processing operations may be performed by the processing algorithm 124D (FIG. 1).

In some embodiments, in addition to detection of HILN, the characterization may include segmentation of the specimen container 202, the carrier 214, and/or the specimen 238. From the segmentation data, post processing may be used for quantification of the specimen 238. In some embodiments, characterization of the physical attributes of the specimen container 202 may be determined by the processing algorithm 124D by analyzing the image data. For example, cap color, cap width 234W (FIGS. 2A-2B), cap height 234H, tube width 236W, and tube height 236H may be determined. From these characterizations, the size and/or type of the specimen container 202 may be determined. Moreover, in some embodiments, the second instrument 104B may also determine the type of the cap 234 (FIGS. 2A-2B), which may be used as a safety check and may determine whether a wrong tube type has been used for the ordered tests. The same may be applied to the carrier 214.

As the components in the second instrument 104B age over time, the components may degrade or change their performance. For example, pixels in the imaging devices 352 may degrade or fail over time. The motor 342 may wear over time and become less responsive. The data generated by the sensors may degrade over time. For example, the ability of the acoustic sensor 347 to sense sounds may change over time. The frequencies and/or intensities of light emitted by the light sources 358 may change over time. One or more of these changes may be monitored, such as by the data change identification program 124B to determine whether the processing algorithm 124D needs to be updated. In addition, if the sensors are changed over time, the replacement sensors may output different data responses, which may require updating the processing algorithm 124D.

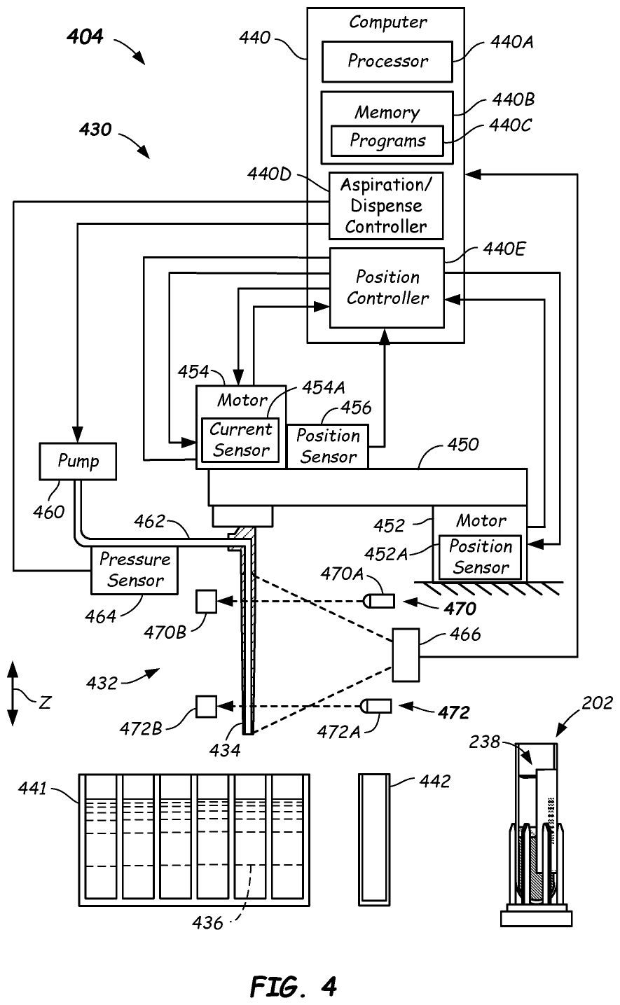

Additional reference is made to FIG. 4, which illustrates a block diagram of an embodiment of an aspiration and dispensing instrument 404, which may be referred to as an aspiration module. The aspiration and dispensing instrument 404 may be implemented in one or more of the instruments 104 (FIG. 1) of the system 100 (FIG. 1). Other embodiments of the aspiration and dispensing instrument 404 may be used in the system 100 (FIG. 1).

The aspiration and dispensing instrument 404 may aspirate and dispense specimens (e.g., specimen 238), reagents, and the like to enable the instruments 104 (FIG. 1) to perform chemical analyses on the specimen 238. The aspiration and dispensing instrument 404 may include a robot 430 that is configured to move a pipette assembly 432 within the aspiration and dispensing instrument 404. In the embodiment of FIG. 4, a probe 434 of the pipette assembly 432 is shown preparing to aspirate a reagent 436 from a reagent packet 441. The specimen container 202 is shown in FIG. 4 with the cap 234 (FIG. 2A) removed, which may have been performed by a decapping instrument (not shown). The pipette assembly 432 may be configured to position the probe 434 so as to aspirate a serum or plasma from the specimen 238. The reagent 436, other reagents, and a portion of the specimen 238 may be dispensed into a reaction vessel, such as a cuvette 442. The cuvette 442 may be made of a material that passes light for photometric analysis by one or more imaging devices as described herein.

Some components of the aspiration and dispensing instrument 404 may be electrically coupled to a computer 440. In the embodiment of FIG. 4, the computer 440 may include a processor 440A and memory 440B. Programs 440C may be stored in the memory 440B and may be executed by the processor 440A. The computer 440 may also include a position controller 440E and an aspiration/dispense controller 440D that may be controlled by programs, such as the programs 440C stored in the memory 440B. In some embodiments, the computer 440 and the components therein may be implemented in the computer 120 (FIG. 1). The position controller 440E and/or the aspiration/dispense controller 440D may be implemented in separate devices (e.g., computers) in some embodiments.

The programs 440C may include algorithms that control and/or monitor components within the aspiration and dispensing instrument 404, such as the position controller 440E and/or the aspiration/dispense controller 440D. As described herein, one or more of the components may include one or more sensors that may be monitored by one of the programs 440C. The programs 440C also may perform self-test routines on the sensors. The results of the self-test routines may be transmitted to the computer 120 (FIG. 1) or the processing algorithm 124D. One or more of the programs 440C may include a processing algorithm similar to the processing algorithm 124D (FIG. 1) that analyzes data generated by the aspiration and dispensing instrument 404.

The robot 430 may include one or more arms and motors that are configured to move the pipette assembly 432 within the aspiration and dispensing instrument 404. In the embodiment of FIG. 4, the robot 430 may include an arm 450 coupled between a first motor 452 and the pipette assembly 432. The first motor 452 may be electrically coupled to the computer 440 and may receive instructions generated by the position controller 440E. The instructions may instruct the first motor 452 as to direction and speed of the first motor 452. The first motor 452 may be configured to move the arm 450 to enable the probe 434 to aspirate and/or dispense specimens and/or reagents as described herein. The first motor 452 may include or be associated with a position sensor 452A that is configured to determine the position of the arm 450. Sensor data generated by the position sensor 452A may be transmitted to the computer 440 and/or the processing algorithm 124D.

A second motor 454 may be coupled between the arm 450 and the pipette assembly 432 and may be configured to move the probe 434 in a vertical direction (e.g., a Z-direction) to aspirate and/or dispense liquids as described herein. The second motor 454 may move the probe 434 in response to instructions generated by the programs 440C. For example, the second motor 454 may enable the probe 434 to enter into and recede from the specimen container 202, the cuvette 442, and/or the reagent packet 441. Liquids may then be aspirated and/or dispensed as described herein. The second motor 454 may include or be associated with a current sensor 454A that is configured to measure current drawn by the second motor 454. Sensor data (e.g., measured current) generated by the current sensor 454A may be transmitted to the computer 440 and/or the processing algorithm 124D.

The aspiration and dispensing instrument 404 may include a plurality position sensors. In the embodiment of FIG. 4, a position sensor 456 is mechanically coupled to the robot 430. In some embodiments, the position sensor 456 may be coupled to other components in the aspiration and dispensing instrument 404. The position sensor 456 may be configured to sense positions of one or more components of the robot 430 or other components within the aspiration and dispensing instrument 404, such as the pipette assembly 432. In the embodiment of FIG. 4, the position sensor 456 may measure the position of the arm 450, the pipette assembly 432, and/or the probe 434. The position data may be transmitted to the computer 440 and/or the processing algorithm 124D (FIG. 1).

The aspiration and dispensing instrument 404 may also include a pump 460 mechanically coupled to a conduit 462 and electrically coupled to the aspiration/dispense controller 440D. The pump 460 may generate a vacuum or negative pressure (e.g., aspiration pressure) in the conduit 462 to aspirate liquids. The pump 460 may generate a positive pressure (e.g., dispense pressure) in the conduit 462 to dispense liquids.

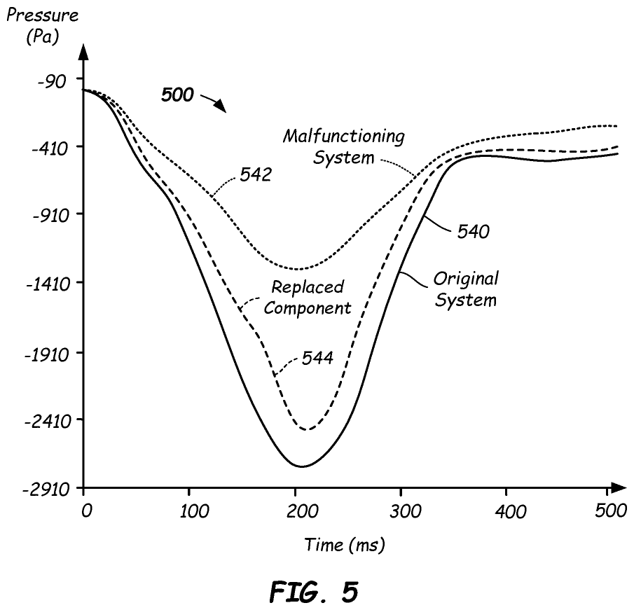

A pressure sensor 464 may be configured to measure pressure in the conduit 462 and generate pressure data indicative of the pressure. In some embodiments, the pressure sensor 464 may be configured to measure aspiration pressure and generate pressure data. In some embodiments, the pressure sensor 464 may be configured to measure dispense pressure and generate pressure data. The pressure data may be in the form of a pressure trace as a function of time and as described with reference to FIG. 5 below. The pressure data ultimately may be transmitted to the computer 120 (FIG. 1) for processing by the processing algorithm 124D. The pressure traces may change as a function of time or when one or more components of the aspiration and dispense instrument 404 are replaced.

Additional reference is made to FIG. 5, which is a graph 500 illustrating pressure traces of the pipette assembly 432 measured by the pressure sensor 464. In the embodiment of FIG. 5, the pressure traces show pressures in the pipette assembly 432 during aspiration operations. An original pressure trace 540 is an example from the original aspiration and dispensing instrument 404 that may include all the original components. In some embodiments, the processing algorithm 124D (FIG. 1) may have been trained based on the original pressure trace 540.

A malfunctioning system may be indicated by the pressure trace 542 and may be due to one or more malfunctioning components, such as the pressure sensor 464 and/or the pump 460. For example, the pressure trace 542 shows a low vacuum, which may be indicative of the pump 460 being unable to generate adequate vacuum for aspiration or a leak in the conduit 462.

FIG. 5 also illustrates a graph that illustrates a pressure trace 544 that may be the result of a replaced component or a worn component. In the embodiment of FIG. 5, the replaced component may not function as well or may function differently than the original component. Examples of replaced components include the pump 460, the pressure sensor 464, and the conduit 462. In some embodiments, original equipment components may not be available, so other aftermarket components may have to be used as replacement components. In some embodiments, the pressure trace 544 may be due to one or more worn or aged components in the aspiration and dispensing instrument 404. The accumulation of data constituting the pressure traces may be the data distribution described herein.

Referring again to FIG. 4, the aspiration and dispense instrument 404 may include an imaging device 466 configured to capture images of the probe 434. For example, the probe 434 may be transparent so the imaging device 466 may capture images of liquids located in the probe 434. The captured images may comprise image data that is transmitted to and analyzed by the computer 440 and/or the processing algorithm 124D (FIG. 1). The programs 440C or the processing algorithm 124D may analyze the image data to determine the quality of the liquid in the probe 434. For example, the programs 440C or the processing algorithm 124D may determine whether the liquid in the probe 434 has bubbles. Images captured by the imaging device 466 may be analyzed by the data change identification program 124B to determine if the processing algorithm 124D described herein needs to be updated. For example, as the imaging device 466 wears or is replaced, the characteristics of the captured images may change, which may require changes to the AI model in the processing algorithm 124D.

In some embodiments, the aspiration and dispense instrument 404 may include one or more other imaging devices implemented as one or more optical sensors that may be configured to sense liquids in the probe 434. In the embodiment of FIG. 4, the aspiration and dispense instrument 404 may include a first optical sensor 470 and a second optical sensor 472.

The first optical sensor 470 may include a first transmitter 470A and a first receiver 470B. The first transmitter 470A may include a light source, such as a laser or light-emitting diode (LED) that is configured to transmit light through the probe 434. The light passing through the probe 434 is received by the first receiver 470B. The second optical sensor 472 may be identical or substantially similar to the first optical sensor 470 and may be located vertically spaced from the first optical sensor 470. The second optical sensor 472 may include a second transmitter 472A and a second receiver 472B. The optical sensors 470, 472 may measure the height of the liquid in the probe 434. Data generated by the first receiver 470B and the second receiver 472B may be processed by the processing algorithm 124D and data change identification program 124B to determine if the data has changed and whether the processing algorithm 124D needs to be updated as described herein.

Should, e.g., components of the first optical sensor 470 degrade over time, the processing algorithm 124D may need to be updated. In addition, should components of the first optical sensor 470 be replaced with devices having different optical characteristics, the processing algorithm 124D may have to be updated to reflect the different optical characteristics.

The system 100 may have different instruments than those described above. Data collected from the instruments 104 may be input into the processing algorithm 124D, which may include an AI model that performs analyses and determines characteristics of the specimen 238, the specimen containers 102, and/or the carriers 114. In some embodiments, the AI model may determine whether the system 100 is in need of maintenance or is ready to fail. In other embodiments, the AI model may be implemented in other programs that may be local to or remote from the system 100.

The AI models that are implemented in the system 100, such as in the processing algorithm 124D, may be trained, engineered, and/or evaluated on large data sets to be robust in a variety of conditions. For example, the AI models may be able to identify different types of specimen containers and perform various analyses on a plurality of different specimen types. The systems, with the AI models, are often deployed for several years. During this time, changes in data distributions inevitably occur. These changes may be due to hardware updates as described herein, changes in software versions, changes in specimen containers 102, such as changes in characteristics of the tube 236, changes in the label 240 or other indicia affixed to the specimen containers 102, different assay protocols, and other reasons. These changes may require the processing algorithm 124D to be updated regularly over time. The system 100 described herein is configured to update the processing algorithm 124D and/or other algorithms based on or including machine learning or AI models to accommodate the changes.

Triggering updates and updating conventional systems may be very cumbersome. In some conventional systems, the need for updating is only discovered while troubleshooting a failure in the system, which means the system has failed instead of having a preemptive update applied. When it is found that an update in a conventional system is necessary, a subsequent effort is triggered to acquire data required to implement the update. This data may then be used by an engineering team to update the AI model(s) to cure the system failures. In some embodiments, the AI models needs to be re-trained to accommodate the updates. The above-described workflow may be very expensive and time-consuming.

The system 100 described herein may detect changes in data (e.g., data distribution) or system usage and update itself to accommodate those changes. For example, the system 100 may retrain the processing algorithm 124D, such as an AI model therein, based on the detected changes in the data. In some embodiments, the system 100 may be capable of detecting changes that could affect performance of one or more analyses and may update the processing algorithm 124D in response to detecting the changes. For example, the data change identification program 124B may compare initial data on which the processing algorithm 124D was trained and determine if new data entered into, generated by, and/or output from the system 100 is capable of being processed by the processing algorithm 124D based on the training using the initial data. If not, an update may be initiated as described herein.

An embodiment of the updating process is summarized below followed by more detailed embodiments. When the processing algorithm 124D is to be updated, the system 100 may acquire specific data and the corresponding labels that may be used to update the processing algorithm 124D. This data is referred to herein as the identified data. In some embodiments, the system 100 may be updated or adapted to changes based on site-specific data and the corresponding labels.

The identified data may include any data input, generated by, and/or output by the system 100 or the instruments 104. Data input to the system 100 that may be identified data includes, but is not limited to, data related to the types of assays that are to be performed, the times the assays are to be performed, the types of specimens received in the system, the types of the specimen containers 102, various inputs received from the user of the system 100 such as via the workstation 128, and other data. Data input to the system 100 that may be identified data also may include data received via the LIS 130. Data generated by the system 100 that may be identified data generated by the system 100 includes, but is not limited to, data generated by any of the sensors in the system 100. Thus, the identified data may include image data, acoustic data, temperature data, pressure traces, and other data. The identified data may also include data generated by any of the programs in the system 100, such as the data processing program 124A, the processing algorithm 124D, and other programs and algorithms. Data output by the system 100 that may be considered identified data includes, but is not limited to, data output to the LIS 130, results of analyses performed on specimens, outputs of instruments that perform analyses on specimens, and other related data.

When the change in data or data distribution is identified, data instances pertaining to the change in the data distribution are aggregated. Thus, when there is a change in the data, instances of that data are aggregated, such as by the data aggregating program 124C. For example, if the tube identification has changed, the data aggregating program 124C may aggregate data of the “new” tubes or tubes not identified by the initial processing algorithm or related program. In another example, if the identified data are pressure traces (FIG. 5), then the pressure traces may be aggregated, such as by the data aggregating program 124C.

Data from a subset of aggregated data is selected, which may be referred to as data sampling. This data is referred to as subset data and is used to update the processing algorithm 124D. The data selecting or data sampling may include determining which data should be incorporated in the updated AI model to avoid divergence or “catastrophic forgetting.” Divergence occurs when the updated AI model performs worse than the initial AI model on the validation dataset. Divergence may be indicative of underfitting, which occurs when the updated AI model is incapable of processing new data. For example, the updated processing algorithm 124D may not be able to analyze data properly. Catastrophic forgetting occurs when the revised AI model is overfitted to the subset data used to retrain the AI model, so the updated AI model is no longer able to perform well on the validation dataset. Neither divergence nor catastrophic forgetting may be acceptable. Catastrophic forgetting restricts the range of improvements that can be made to the AI model. Divergence may prevent the updated AI model from meeting the requirements of regulatory clearance obtained using the original validation data. Therefore, updates may only be applied when the updates are likely to improve the system 100 and not cause catastrophic forgetting or divergence.

In some embodiments, updating the AI model may require updating the capacity of the AI model (e.g., model capacity) such as by adding residual layers or model weights, which may be achieved by deciding which data samples to use for backpropagation. With regard to model weights, a deep network, such as a variational autoencoder, can be trained to determine if the provided sample causes catastrophic forgetting or divergence.

The above-described methodologies may enable the system 100 to readily update the AI model(s) to address cases that may not have been handled well by the initial AI model. For example, the initial processing algorithm may try to process clinical outlier data the same way as routine data, which can potentially lead to degeneration of the AI model as it uses the data for upgrading. The degradation may be avoided by enabling the updated AI model to have access to a validation dataset that the performance of the updated AI model is evaluated against. If a divergence occurs, the updated AI model may be rolled back to an older or previous AI model. In some embodiments, instead of updating the AI model as the data becomes available, the system 100 can have access to a fixed size storage, where subset data can be stored. In such embodiments, the updates can be applied together with a batch of data, or the data can be sent to a location where others may train the AI model and later send the updated AI model to the system 100. The updated AI model sent from the others may then be installed in the processing algorithm 124D.

As described above, the updated AI model may be tested against a validation dataset, which may be or include validation data collected across multiple systems and/or across multiple instruments in one or more systems. The validation data may also be collected from multiple laboratories, including laboratories worldwide. In some embodiments, the validation dataset may include validation data that was used for regulatory approval and to maintain the integrity of the initial AI model performance. In some embodiments, additional data (e.g., data samples) may be added to the validation dataset to accommodate the hardware/software changes. In some embodiments, the changes may be changes that could affect large numbers of systems, such as across large geographic regions, and not just specific systems or a small geographic region. In some embodiments, the validation dataset may be compressed and/or encrypted. In some embodiments, the validation dataset may be deployed in the system 100, in the cloud, or at a remote site.

The ground truth for the validation dataset may come from different sources. In some embodiments, the ground truth may come from one or more secondary resources, such as a gold standard instrument or system. In some embodiments, the ground truth may be automatically generated using an existing system that is trained or via self-supervision training techniques. In some embodiments, the validation dataset may be manually generated or annotated.

In some embodiments, a continuous learning workflow may be operating in the background, wherein the AI model is updated using any of the above-mentioned scenarios. Operating in the background includes situations where the system 100 continues to analyze specimens while the AI model is updating. In some embodiments, the AI model update may be triggered as a software patch for immediate use in the system 100. In some embodiments, the software patch may be applied upon confirmation, such as from a system user (e.g., a clinician or technician) on a user interface, such as by the workstation 128. The user may have the option to set or reset the AI model to a previous version, such as an original or factory-delivered model. In other embodiments, the user may send an updated AI model to an external party, such as developers or a factory, for validation. In some embodiments, the system 100 can suggest an update to the AI model if changes in data are detected. The external party may then perform the validation process on the suggested AI model. In some embodiments, only validated AI models can be patched to the system 100.

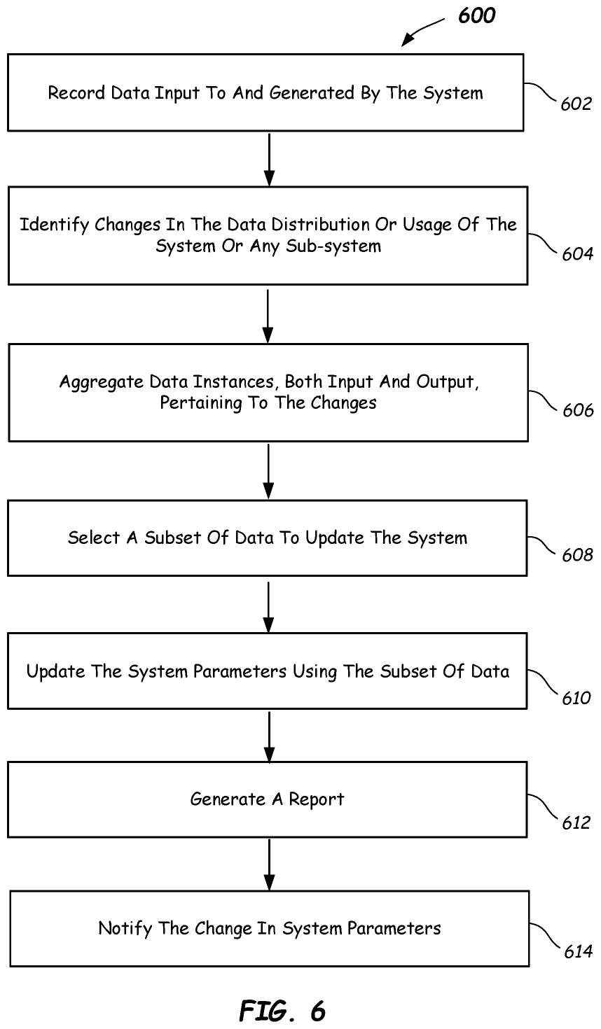

Reference is made to FIG. 6, which is a flowchart illustrating an embodiment of a method 600 of updating AI models (e.g., the processing algorithm 124D) in a laboratory diagnostic system, such as the system 100 described herein. In some embodiments, the method 600 of updating algorithms may be automated. The method 600 may be implemented in the computer 120, such as by one or more of the programs in the memory 124. In some embodiments, the AI model(s) may be implemented and/or stored in processing algorithm 124D.

In a first block 602 the method 600 includes recording data input to and/or generated by the system 100 or any subsystem. The data may be recorded, for example, in the data processing program 124A. Subsystems may include any of the instruments 104 and/or devices associated with and/or coupled to the system 100. This data may, as an example, be stored in the memory 124. The data input to the system 100 may include data received from the LIS 130 and/or the HIS 132. The data input to the system 100 may include types of assays being ordered, times that certain assays are ordered, medical professionals ordering certain assays, and other order information.

The data input to the system 100 may also include characteristics of the specimen containers 102. Referring to FIGS. 2A-2B, the characteristics of the specimen containers 102 may include shape, color, and/or physical dimensions of the caps (e.g., cap 234). Different caps having different characteristics may be used within the system 100 and may change over time. The characteristics may also include shape and/or physical dimensions of the tube 236. Different tubes having different shapes and/or physical dimensions may be used in the system 100 and may change over time. The characteristics may also include the label 240 and the identification information 242. Different versions of labels and identification information may be used in the system 100 and may change over time.

The data generated by the system 100 may include data generated by sensors and/or data generated by analyzing specimen containers 102 and/or specimens located therein as described herein. In some embodiments, the data generated by the system 100 may include data generated by the processing algorithm 124D.

The method 600 may include, in block 604, identifying changes in the data distribution or usage of the system 100 or any subsystem. Although the following description describes changes in the data, in some embodiments, there may be a single change in the data. The changes in the data may be differences from the data on which the initial AI model in the processing algorithm 124D was trained. For example, the data may be compared to the validation dataset that was used to train the AI model.

The method 600 may include, in block 606, aggregating data instances, both input and output, pertaining to the changes identified in block 604. By aggregating the data instances, the system 100 or program performing the method 600 may determine a status of the system 100, including the sensors, instruments 104, and analyses results when the change in data occurs. Aggregating the data also enables one or more subsets of data to be sampled or selected to update the AI algorithm.

The method 600 may include, in block 608, selecting a subset of data to update the system 100. The subset of data is from the aggregated data and is referred to as the subset data. The subset data may include, but is not limited to, images (image data), text, and/or any sensor data along with the ground truth associated with the subset of data.

The method 600 may include, in block 610, updating the system parameters (e.g., the parameters of the processing algorithm 124D) using the subset data. Updating the system parameters may include updating the processing algorithm 124D using the subset data, which may also include updating the AI model. In some embodiments, the system 100, such as the computer 120, may update or retrain the initial AI model to the updated AI model. The updated AI model may then be transmitted back to the computer 120 where the processing algorithm 124D or another program that stores the AI model is updated.

The method 600, in block 612, may generate a report in response to updating the processing algorithm 124D. In some embodiments, the report may include explanations of the updated AI model. The report may, in some embodiments, be output by the workstation 128. The report may be compliant with local laboratory regulations and may certify that the updated AI model and/or the updated processing algorithm 124D are compliant with local laboratory regulations.

The method 600, in block 614, may notify the change in the AI model and/or the processing algorithm 124D. In some embodiments, a user of the system 100 may reject the updates in response to the notification. Accordingly, the system 100 may be configured to replace the updated AI model and/or the processing algorithm with a previous version.

Having described overviews of various systems and methods of updating processing algorithms, specific examples are now described. Some of the examples explain a continuous training or learning method of the AI model.

The following example relates to tube type classification and/or categorization performed by the one or more of the instruments 104 in the system 100 using one or more programs in the memory 124. The goal of the classification is to categorize each of the sample tubes (e.g., tube 236) into plain tube, capped tube, or tube top sample cup (TTSC). Characterization enables one or more sample handler robotic grippers to adopt an associated motion profile for optimizing tube handling speed. In some embodiments, each laboratory may have specific preferences on the selections of tubes used in their appropriate systems. Pre-trained (e.g., initially-trained) AI models may not have been trained with a particular sample tube make and model and, thus, the AI models may not be able to achieve an expected accuracy. Accordingly, there may be discrepancies between tubes used in the system 100 and identification performed by the processing algorithm 124D.

The system 100 may utilize several methods of determining the discrepancy between the tubes used by the system 100 and the identification as determined by the processing algorithm 124D. Site-specific metrics, such as those specific to the system 100 may be collected and compared with expected statistics for the system 100. For example, if the plain tube utilization of the system 100 is about 85% and the AI model of the processing algorithm 124D classifies most of the tubes as TTSC, the existing AI model may not fit the site-specific data. The deviation may trigger on-site data collection and/or model updating to update the AI model as described herein.

Another way to find the discrepancy is to utilize a confidence value generated from the AI model during classification. If the AI model generates a low confidence value during classification, the low confidence may indicate that the AI model is uncertain about the tube classification and updating the AI model may be needed. In some embodiments, the statistics of confidence value may also be used as an indicator of how difficult it is to perform the site-specific adaptation for the AI model, where low confidence may imply that more resources (e.g., a large amount of training samples) and efforts are needed to update the AI model.

Data related to the tubes input to the system 100 may be stored or processed by the data processing program 124A. The processing algorithm 124D may process data received from one or more of the instruments 104 to determine tube types. The data change identification program 124B may detect a change in the tube types either received by the system 100 or determined by the processing algorithm 124D.

The data related to the tubes that are not classified may be aggregated by the data aggregating program 124C. The aggregated data may include data related to the tube types that may serve as data to update the AI model in the processing algorithm 124D. A subset of the data may be selected. The subset of data may be tube type data on which the updated AI model may accurately classify the tube types in a validation data set that may include tube types. If the updated AI model correctly classifies the tube types, the processing algorithm 124D may be updated with the updated AI model. In some embodiments, a user of the system 100 may provide an input to the system 100, that causes the system 100 to include the updated AI model in the processing algorithm 124D. In some embodiments, the system 100 may generate a report describing the updated AI model and/or abilities of the updated AI model to classify tube types.

Another example relates to the pressure traces of FIG. 5. The AI model may have been trained based on the original pressure trace 540. Over time, the pressure trace of the aspiration and dispensing instrument 404 may change to the pressure trace 542. The pressure trace 542 may be acceptable and may be a result of aging or changed components of the aspiration and dispensing instrument 404. One or more portions of the pressure trace 542 may be the aggregated data and used to update the AI model as described above.

In some embodiments, text reports and/or logs generated by the system 100 may be processed automatically by an AI model, such as the AI model in the processing algorithm 124D, to analyze the system 100 to make sure the system 100 is performing as expected. For example, the AI model may detect whether there is any sort of anomaly in the system 100. These embodiments of the system 100 may utilize natural language processing techniques to reach conclusions as the text reports or logs can sometimes have user input text, such as sentences or paragraphs that explain the conditions. The system 100 also may use calibrated sensor values to detect signs of abnormalities in the system 100. If the sensors and/or related hardware are replaced, then the system may need to be recalibrated, and the recalibrated values may be the detected and/or aggregated data and may be used to retrain the AI model.

In some embodiments, the system 100 may include a predictive maintenance program that uses heterogenous data from various sensors, such as the sensors described herein. The program may collectively analyze data generated by the various sensors, including sensor measurements from individual submodules, to determine the state of the system 100 and forecast potential system failures. An AI model, such as a deep generative network, may be used to encode the sensor data obtained during one or more operations of the instruments 104 as a specific fingerprint representative of the dynamics of the operations of the system 100. The program may use another AI model to collectively analyze these fingerprints over a period to forecast potential failures of the system 100. As the sensors change, the AI model may be updated as described herein to reflect the changes in the sensors and to accurately predict system failures.

In some embodiments, selecting a subset of the aggregated data may include selecting a ground truth. In some embodiments, updating (e.g., training) the processing algorithm 124D or the AI model therein includes training the processing algorithm 124D on data input to the system 100, wherein the data input to the system 100 has population statistics. Input data statistics of data input to the system 100 may be calculated and a change in data distribution may be related to changes in the input data statistics. These changes may be identified by the data change identification program 124B and used to update the processing algorithm 124D as described herein. In some embodiments, the change in data distribution may include at least one change in at least one interaction between a user of the system 100 and the system 100.

In some embodiments, aggregating data instances includes aggregating input data to a first subsystem (e.g., an instrument or a component of an instrument) in the system 100 with expected output data of the first subsystem. The expected output data of the first subsystem may be obtained using measurements generated by a second subsystem. In some embodiments, the expected output data of the first subsystem is obtained by at least one interaction between a user interacting with the system 100. In some embodiments, selecting a subset of the aggregated data includes selecting at least two subsets of data, wherein a first subset of data is used to compute parameters of the processing algorithm 124D and a second subset of data is used to evaluate a plurality of parameter sets and selecting one or more algorithm parameter sets in response to the evaluating. In some embodiments, parameters of the updated processing algorithm 124D are determined using a conjunction of the aggregated data and a prior training dataset used to train the initial AI model or a previous version of the AI model.

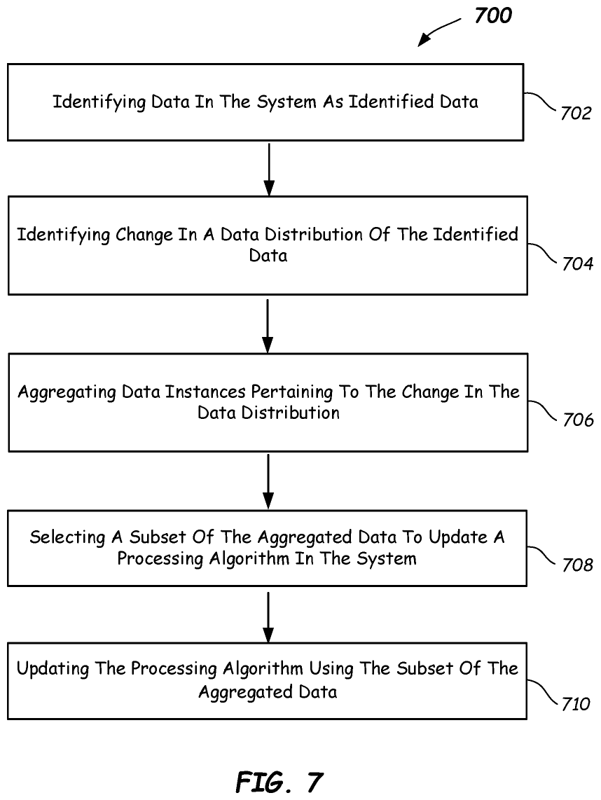

Reference is now made to FIG. 7, which is a flowchart illustrating a method 700 of operating a diagnostic laboratory system (e.g., system 100). The method 700 includes, in block 702, identifying data in the system as identified data. The method includes, in block 704, identifying change in a data distribution of the identified data. The method includes, in block 706, aggregating data instances pertaining to the change in the data distribution. The method includes, in block 708, selecting a subset of the aggregated data to update a processing algorithm (e.g., processing algorithm 124D) in the system. The method 700 includes, in block 710, updating the processing algorithm using the subset of the aggregated data.

While the disclosure is susceptible to various modifications and alternative forms, specific method and apparatus embodiments have been shown by way of example in the drawings and are described in detail herein. It should be understood, however, that the particular methods and apparatus disclosed herein are not intended to limit the disclosure but, to the contrary, to cover all modifications, equivalents, and alternatives falling within the scope of the claims.

What is claimed is:

1. A method of operating a diagnostic laboratory system, comprising:
identifying data in the system as identified data;
identifying change in a data distribution of the identified data;
aggregating data instances pertaining to the change in the data distribution;
selecting a subset of the aggregated data to update a processing algorithm in the system; and
updating the processing algorithm using the subset of the aggregated data.

2. The method of claim 1, wherein the data in the system is data input to the system.

3. The method of claim 1, wherein the data in the system is data generated by the system.

4. The method of claim 1, further comprising generating a notice indicating that the processing algorithm has been updated.

5. The method of claim 1, wherein the identifying data in the system comprises identifying data input to the system.

6. The method of claim 5, wherein the data input to the system includes at least one of assay type to be performed on a specimen, date that an assay is performed on the specimen, and time that the assay is performed.

7. The method of claim 1, wherein the identifying data in the system comprises data output from the system.

8. The method of claim 1, wherein the identifying data in the system comprises identifying data generated by at least one sensor located in the system.

9. The method of claim 1, wherein the identifying data in the system comprises identifying image data generated by at least one imaging device located in the system.

10. The method of claim 1, wherein the identifying data in the system comprises identifying at least one of: acoustic data, image data, and temperature data.

11. The method of claim 1, wherein the selecting a subset of the aggregated data comprises selecting a ground truth.

12. The method of claim 1, further comprising:
training the processing algorithm on data input to the system; and
calculating input data statistics of data input to the system,
wherein the change in data distribution is related to changes in the input data statistics.

13. The method of claim 1, wherein the change in data distribution is at least one change in at least one interaction between a user of the system and the system.

14. The method of claim 1, wherein the aggregating data instances comprises aggregating input data to a first subsystem in the system with expected output data of the first subsystem, wherein the expected output data of the first subsystem is obtained using measurements generated by a second subsystem.

15. The method of claim 1, wherein the aggregating data instances comprises aggregating input data to a first subsystem in the system with expected output data of the first subsystem, wherein the expected output data of the first subsystem is obtained by at least one interaction between a user interaction of the system and the system.

16. The method in claim 1, where the selecting a subset of the aggregated data comprises selecting at least two subsets of data, wherein a first subset of data is used to compute parameters of the processing algorithm and a second subset of data is used to evaluate a plurality of parameter sets and to select one or more algorithm parameters in response to the evaluating.

17. The method in claim 1 where parameters of the updated processing algorithm are determined using a conjunction of the aggregated data in addition to a prior training dataset.

18. The method of claim 1, further comprising testing the updated processing algorithm on a validation dataset.

19. The method in claim 1, further comprising continuously updating the processing algorithm.

20. A diagnostic laboratory system, comprising:
a computer comprising:
a processing algorithm configured to analyze data; and
a program configured to:
identify data in the system as identified data;
identify change in a data distribution of the identified data;
aggregate data instances pertaining to the change in the data distribution;
select a subset of the aggregated data to update the processing algorithm in the system; and
update the processing algorithm using the subset of the aggregated data.

21. A method of operating a diagnostic laboratory system, comprising:
identifying data in the system as identified data;
identifying change in a data distribution of the identified data;
aggregating data instances pertaining to the change in the data distribution;
selecting at least two subsets of data;
evaluating at least one algorithm parameter of a processing algorithm using a first subset of data;
evaluating at least one of the algorithm parameters using a second subset of data;
selecting one or more of the algorithm parameters in response to the evaluating; and
updating the processing algorithm using the one or more selected algorithm parameters.
